(19) United States
(12) Patent Application Publication
(10) Pub. No.: US 2024/0285261 A1
(43) Pub. Date: Aug. 29, 2024

(54) ULTRASOUND APERTURE COMPOUNDING METHOD AND SYSTEM

(71) Applicant: BFLY Operations, Inc, Burlington, MA (US)

(72) Inventors: Karl Thiele, St. Petersburg, FL (US); Joseph Feld, New York, NY (US)

(73) Assignee: BFLY Operations, Inc, Burlington, MA (US)

(21) Appl. No.: 18/590,020

(22) Filed: Feb. 28, 2024

Related U.S. Application Data
(60) Provisional application No. 63/448,939, filed on Feb. 28, 2023.

(51) Int. Cl. A61B 8/08; A61B 8/00; G06T 5/50; G06T 5/70

(52) U.S. Cl. CPC A61B 8/5269; A61B 8/4494; A61B 8/5207; A61B 8/5246; G06T 5/50; G06T 5/70; G06T 2207/10132; G06T 2207/20216

(57) ABSTRACT

Systems that include an array of ultrasound transducers divided into two or more sub-arrays where for example, a one or two dimensional array, with a long axis in a lateral direction, may be divided in half. The system may include a different beamformer for each sub-array. Each sub-array may define independent and spatially separated sub-apertures. The spatial separation of the two sub-apertures allows for aperture compounding to reduce speckle because the received ultrasound waves at each sub-aperture are propagating in a different direction with respect to each other. This may allow the point spread function for the ultrasound signals corresponding to each sub-aperture to be decorrelated for reducing speckle. The speckle can be reduced by averaging the ultrasound signal from each of the sub-apertures, and a higher resolution can be maintained by also using the signal from the full aperture.

This application claims priority to U.S. Provisional Application No. 63/448,939 filed 28 Feb. 2023 and entitled ULTRASOUND APERTURE COMPOUNDING METHOD AND SYSTEM, the contents of which are hereby incorporated by reference in their entirety.

### BACKGROUND

Ultrasound imaging has a wide range of applications in the medical and scientific fields for diagnosis, treatment, and studying of internal objects, within a body, such as internal organs or developing fetuses. An ultrasound probe typically includes an array of transducers that transmit and receive ultrasound signals that are used for these imaging techniques. Speckle is a type of artifact or noise that is present in ultrasound imaging due to coherent interference from ultrasound waves scattered by a heterogeneous medium. Speckle manifests as randomly oriented light and dark areas in an ultrasound image that can result in reduced interpretable detail and contrast of features in the image. One way to mitigate speckle in ultrasound imaging is known as “spatial compounding” where the operator of the ultrasound probe takes two or more frames of the same region such that the probe is tilted at a different angle for each of the two images. By tilting the probe, the propagation directions of the ultrasound waves are tilted with respect to each other for each of the frames. This leads to a decorrelation of the point-spread functions of each frame such that the frames can be averaged to produce a final ultrasound image where the speckle is reduced. However, spatial compounding as described above, requires two or more transmit events where the operator of the probe physically moves the probe between the two frames, which can lead to slower imaging and other potential degradations of the image.

### BRIEF DESCRIPTION OF DRAWINGS

FIG. 1 shows a block diagram of an aperture compounding ultrasound system, according to one or more embodiments.

FIG. 2 shows a flow chart for a method for aperture compounding in ultrasound imaging, according to one or more embodiments.

FIG. 3A shows a simulated transmission point spread function for an aperture compounding ultrasound system, according to one or more embodiments.

FIG. 3B shows a simulated reception point spread function for an aperture compounding ultrasound system, according to one or more embodiments.

FIG. 3C shows a simulated round trip point spread function for an aperture compounding ultrasound system, according to one or more embodiments.

FIG. 4A shows the structure of a simulated phantom medium, according to one or more embodiments.

FIG. 4B shows a simulated ultrasound image for a full receive aperture without compounding, according to one or more embodiments.

FIG. 4C shows a simulated ultrasound image for a half receive aperture without compounding, according to one or more embodiments.

FIG. 4D shows a simulated ultrasound image for a left half receive aperture without compounding, according to one or more embodiments.

FIG. 4E shows a simulated ultrasound image for a right half receive aperture without compounding, according to one or more embodiments.

FIG. 4F shows a simulated ultrasound image where a left half receive aperture and a right half receive aperture are compounded, according to one or more embodiments.

FIG. 4G shows a simulated ultrasound image where a left half receive aperture, a right half receive aperture, and the full receive aperture are compounded, according to one or more embodiments.

FIG. 5A shows an empirical ultrasound image collected using a whole receive aperture of an ultrasound system without compounding, according to one or more embodiments.

FIG. 5B shows an empirical ultrasound image collected using a left half receive aperture of an ultrasound system without compounding, according to one or more embodiments.

FIG. 5C shows an empirical ultrasound image collected using a right half receive aperture of an ultrasound system without compounding, according to one or more embodiments.

FIG. 5D shows an empirical ultrasound image collected by compounding a left half receive aperture and a right half receive aperture of an ultrasound system, according to one or more embodiments.

FIG. 5E shows an empirical ultrasound image collected by compounding a left half receive aperture, a right half receive aperture, and the full aperture of an ultrasound system, according to one or more embodiments.

FIG. 6 shows a block diagram of an aperture compounding ultrasound system, according to one or more embodiments.

FIG. 7A shows a simulated source reception point spread function for an aperture compounding ultrasound system, according to one or more embodiments.

FIG. 7B shows a simulated destination reception point spread function for an aperture compounding ultrasound system, according to one or more embodiments.

FIG. 7C shows a simulated roundtrip point spread function for an aperture compounding ultrasound system, according to one or more embodiments.

FIG. 8A shows the structure of a simulated phantom medium, according to one or more embodiments.

FIG. 8B shows a simulated ultrasound image for a full receive aperture without compounding, according to one or more embodiments.

FIG. 8C shows a simulated ultrasound image where a left half receive aperture, a right half receive aperture, and the full receive aperture are compounded, according to one or more embodiments.

FIG. 8D shows a simulated ultrasound image where a first and third chunk receive aperture, a second and fourth chunk receive aperture, and the full receive aperture are compounded, according to one or more embodiments.

FIG. 9 shows a block diagram of an example of an ultrasound device according to one or more embodiments.

FIG. 10 shows a schematic block diagram of an example ultrasound system, according to one or more embodiments.

FIG. 11 shows an example handheld ultrasound probe, according to one or more embodiments.

FIG. 12 shows an example wearable ultrasound patch, according to one or more embodiments.

FIG. 13 shows an example ingestible ultrasound pill, according to one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third) may be used as an adjective for an element (i.e., any noun in the application). The use of ordinal numbers is not to imply or create a particular ordering of the elements nor to limit any element to being only a single element unless expressly disclosed, such as by the use of the terms “before,” “after,” “single,” and other such terminology. Rather the use of ordinal numbers is to distinguish between the elements. By way of an example, a first element is distinct from a second element, and the first element may encompass more than one element and may succeed (or precede) the second element in an ordering of elements.

In general, embodiments of the disclosure provide a method, non-transitory computer readable medium (CRM), and system for reducing speckle in ultrasound data. In one or more embodiments, the system may include an array of ultrasound transducers that can be divided into two or more sub-arrays. For example, a one or two dimensional array, with a long axis in a lateral direction, may be divided in half such that one sub-array is entirely on one side of the center of the array and the other sub-array is entirely on the other side of the center of the array in the lateral direction. The system may include a different beamformer for each sub-array so that ultrasound waves detected by each sub-array can be independently beamformed in parallel and output to the host (for example, an attached phone, tablet, or computer) for further processing. Each sub-array defines independent and spatially separated sub-apertures that receive the ultrasound waves. The spatial separation of the two sub-apertures allows for “aperture compounding” to reduce speckle because the received ultrasound waves at each sub-aperture (corresponding to a given focal point in the medium being imaged) are propagating in a different direction with respect to each other. This may allow the point spread function for the ultrasound signals corresponding to each sub-aperture to be decorrelated for reducing speckle. The host may obtain the signals corresponding to each sub-aperture and coherently sum them, effectively reproducing the signal from the full aperture (i.e., corresponding to the full array of transducers). The host may then logarithmically detect each of these three signals (i.e., the signals from each of the sub-apertures and reproduced full aperture signal) and average them to generate the final averaged ultrasound signal. In this way, the speckle can be reduced by averaging the ultrasound signal from each of the sub-apertures, and a higher resolution can be maintained by also using the signal from the full aperture. Because the above process does not require acquiring different frames between which the ultrasound probe is physically moved, the high-resolution, reduced-speckle image can be achieved at a high frame rate/speed.

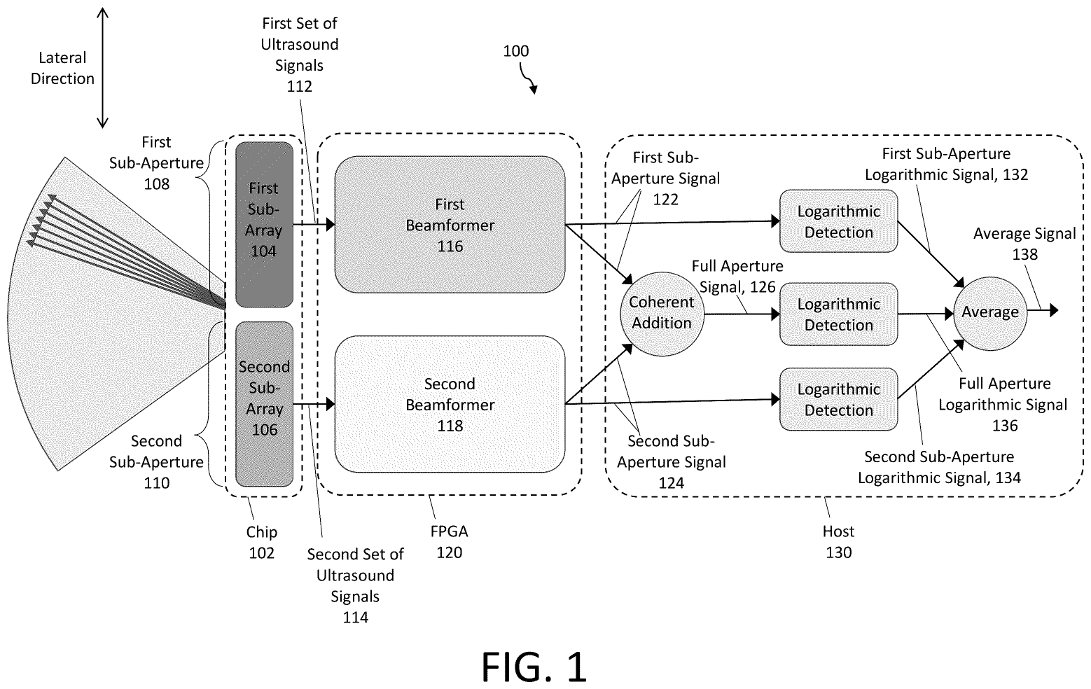

FIG. 1 shows a block diagram of an ultrasound system 100 for reducing speckle in ultrasound data by aperture compounding, according to one or more embodiments. In one or more embodiments, the system 100 includes an ultrasound probe that transmits and/or receives ultrasound waves and converts them into beamformed ultrasound signals or data streams. As will be discussed below, the ultrasound probe may be a handheld probe, an ingestible pill, a wearable patch, or any other suitable form factor for ultrasound imaging. In one or more embodiments, the ultrasound probe includes a chip 102 that has an array of ultrasound transducers that each convert electronic signals into ultrasound waves (transmission) and/or convert ultrasound waves to electronic signals (reception). As will be discussed in further detail below, the ultrasound transducers may be capacitive micromachined ultrasound transducers (CMUTs), piezo micromachined ultrasound transducers (PMUTs), or any other suitable type or implementation of an ultrasound transducer.

In one or more embodiments, the array of the ultrasound transducers is a two-dimensional array (i.e., an m by n array of ultrasound transducers, where n is the number of transducers in the lateral direction, as shown in FIG. 1, and m is the number of ultrasound transducers in a direction perpendicular to the lateral direction). In one or more embodiments, n>m such that the number of transducers in the array is larger in the lateral direction (i.e., the long axis of the array of ultrasound transducers is oriented in the lateral direction). However, the array of ultrasound transducers is not limited to the above description. In other embodiments, the shape of the two-dimensional array may be any other suitable shape and m and n may each be any suitable number. In still other embodiments, the array of ultrasound transducers may also be a one-dimensional array such that the transducers form a line extending substantially in the lateral direction. Therefore, in the case of both the one-dimensional array and the two-dimensional array, the array is distributed in the lateral direction. In the one-dimensional array case, each transducer is distributed along the lateral direction, while in the two-dimensional array case, rows (m) of transducers are distributed along the lateral direction.

Additionally, regardless of whether the array is a one-dimensional or two-dimensional array, the array may be either curved or straight. In other words, the array of transducers may be distributed across a surface that is flat or curved to be either concave or convex, depending on the required specifications for the particular application for the ultrasound system.

In one or more embodiments, the array of ultrasound transducers is divided into two or more sub-arrays (e.g., a first sub-array 104 and a second sub-array 106). The area of the first sub-array 104 defines a first sub-aperture 108, and the area of the second sub-array 106 defines a second sub-aperture 110 over which ultrasound waves that are incident upon the sub-apertures 108 and 110 are detected. In this way, the ultrasound waves that are detected by the first and second sub-apertures 108, 110 are spatially separated from each other at the chip 102. Therefore, ultrasound radiation originating and propagating from any particular point (i.e., the focal point) that is detected at the first sub-aperture 108 is propagating at a different angle with respect to ultrasound radiation originating and propagating from the same point that is detected at the second sub-aperture 110.

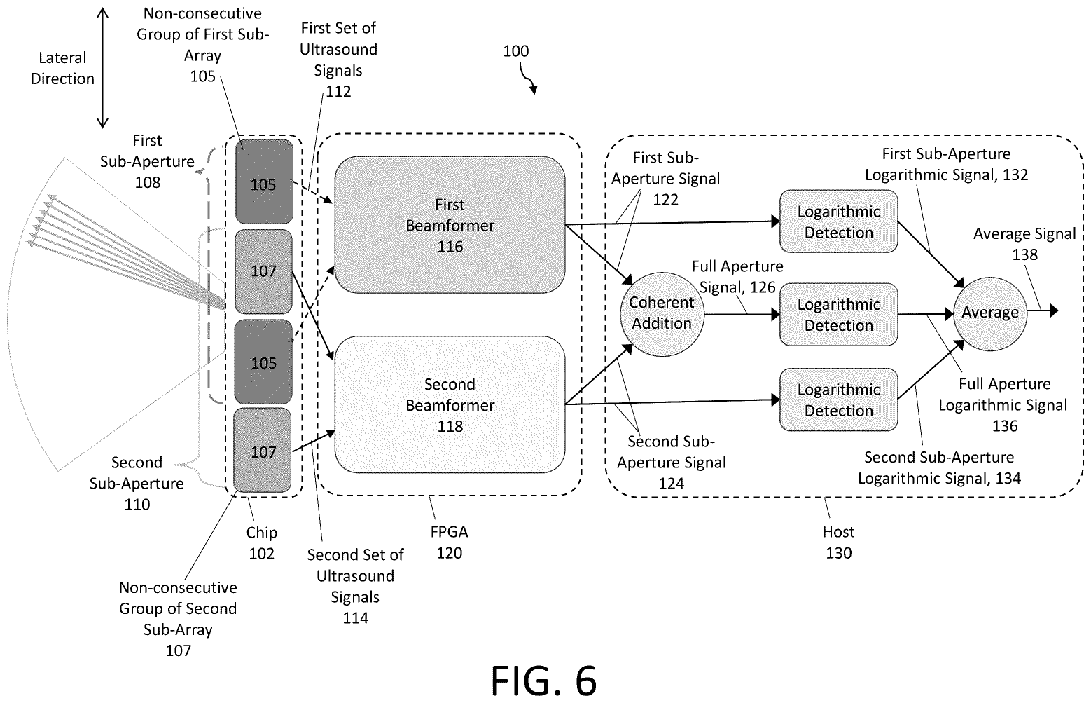

In one or more embodiments, the first and second sub-apertures 108 and 110 are spatially continuous in the lateral direction, as shown in FIG. 1. In other words, the first sub-array 104 and the second sub-array 106 each comprise ultrasound transducers of the array that are consecutive in the lateral direction. However, in other embodiments, the sub-apertures 108, 110 (and therefore the sub-arrays 104, 106) may not be continuous. For example, the sub-apertures 108, 110 may be intermittent (i.e., grouped into smaller portions of the sub-aperture that are not directly adjacent to each other). An example of one configuration including intermittent sub-apertures will be discussed further below with respect to FIGS. 6-8.

In one or more embodiments, the first and second sub-apertures 108 and 110 do not spatially overlap, as shown in FIG. 1. In other words, the sub-arrays 104 and 106 do not share any transducer elements in common. However, the disclosure should not be limited to this case. In other embodiments, the first sub-aperture 108 and the second sub-aperture 110 may partially overlap such that the first sub-array 104 shares some of the ultrasound transducers in common with the second sub-array 106. Whether or not the first and second sub-apertures 108, 110 overlap and to what extent they overlap may be determined by design choices for different ultrasound probes/systems regarding trade-offs between reducing speckle and maintaining a high resolution or optimization of any other applicable parameter or performance metric.

Furthermore, some embodiments of the ultrasound system 100 may include an ultrasound probe that includes circuitry allowing an operator or user to dynamically select between the sizes, degree of overlap, and continuity of the first and second sub-arrays 104, 106 (and therefore also the first and second sub-apertures 108, 110). Additionally, some embodiments of the ultrasound system 100 may include an ultrasound probe that includes circuitry allowing an operator or user to dynamically select the number of sub-arrays into which the array is divided. For example, a user or an operator may be able to select between using only the full array/aperture, dividing the array into two sub-arrays, three sub-arrays, four sub-arrays or more, or a mode where the sub-arrays and the full array are used together to achieve reduced speckle as described further below. In embodiments where these modes are dynamically selectable by a user/operator, the modes may be selected using either a physical switch on the ultrasound probe or in software via the host or computer that is connected to the ultrasound probe.

In one or more embodiments, the first sub-array 104 of transducers converts ultrasound waves incident upon the first sub-aperture 108 into a first set of ultrasound signals 112 that is then transmitted to a first beamformer 116 coupled to the first sub-array 104. The second sub-array 106 of transducers converts ultrasound waves incident upon the second sub-aperture 110 into a second set of ultrasound signals 114 that is transmitted to a second beamformer 118 coupled to the second sub-array 106. The sets of ultrasound signals 112, 114 may include ultrasound signals originating from some or all of the individual ultrasound transducers in the transducer sub-arrays 104, 106. In one or more embodiments, the beamformers 116, 118 are implemented by a field-programmable gate array (FPGA) 120 within the ultrasonic probe.

However, the beamformers may also be implemented in other ways such as using standalone electronic components and/or via dedicated integrated circuitry. These ultrasound signals 112, 114 may be transmitted between the chip 102 and the beamformers 116, 118 by any suitable electronic connection, such as wires, direct contacts between the chip 102 and the FPGA 120, or integrated circuits.

In one or more embodiments, the beamformers 116, 118 each perform “delay-and-sum” beamforming in order to produce ultrasound images. For a given transmit event (i.e., an event where one or more of the transducers transmits ultrasound waves into a medium), the ultrasound waves will be scattered by an object, inclusions, or the medium itself at various points within the medium). These scattered ultrasound waves are detected at the first and second apertures 108, 110 by the first and second sub-arrays 104, 106 and are converted to the electronic ultrasound signals 112, 114. As an example, the first sub-array transmits the first set of ultrasound signals 112 (i.e., the set of ultrasound signals from each transducer in the first sub-array 104) to the first beamformer 116. The first beamformer 116 applies a delay to each signal in the first set of ultrasound signals 112, where the delay applied to each signal, within the first set of ultrasound signals 112, corresponds to a particular focal point within the field of view of the system (i.e., to a particular pixel within the final ultrasound image). Each of the delayed signals are then coherently summed to produce an output signal (referred to as the first sub-aperture signal 122 in this disclosure). The beamformer 116 performs this process using different delays in order generate the output signals corresponding to each different focal point or pixel in the final image. In other words, for every transmit event, the beamformer applies different sets of delays to the detected first set of ultrasound signals 112 in order to generate first sub-aperture signals 122 for every pixel in the final ultrasound image. The second beamformer 118 performs a similar “delay-and-sum” process to the second set of ultrasound signals 114, as that described above. Additionally, in some embodiments the first and second beamformers 116, 118 may perform the “delay-and-sum” process simultaneously in parallel or in successive, separate steps.

The delay applied by the beamformers 116, 118 may be used both in transmission of ultrasound waves and/or in reception of ultrasound waves. In both transmission and reception, the delays applied to the signals for each transducer can be chosen to select both a steering angle of propagation of the ultrasound waves and/or whether and to what extent the ultrasound waves are focused to a focal point. In this way the delays applied to the signals can select a particular focal point within the field of view, as discussed above.

As described above and shown in FIG. 1, one or more embodiments are implemented using two beamformers 116, 118. However, the invention is not limited to implementation with two beamformers. For example, one, two, three, four, or any suitable number of beamformers may be included to optimize for specific design parameters and imaging applications. For example, in some embodiments, as discussed above, more than two sub-arrays/sub-apertures may be employed for a specific application, and an embodiment may include the same number of beamformers as sub-arrays.

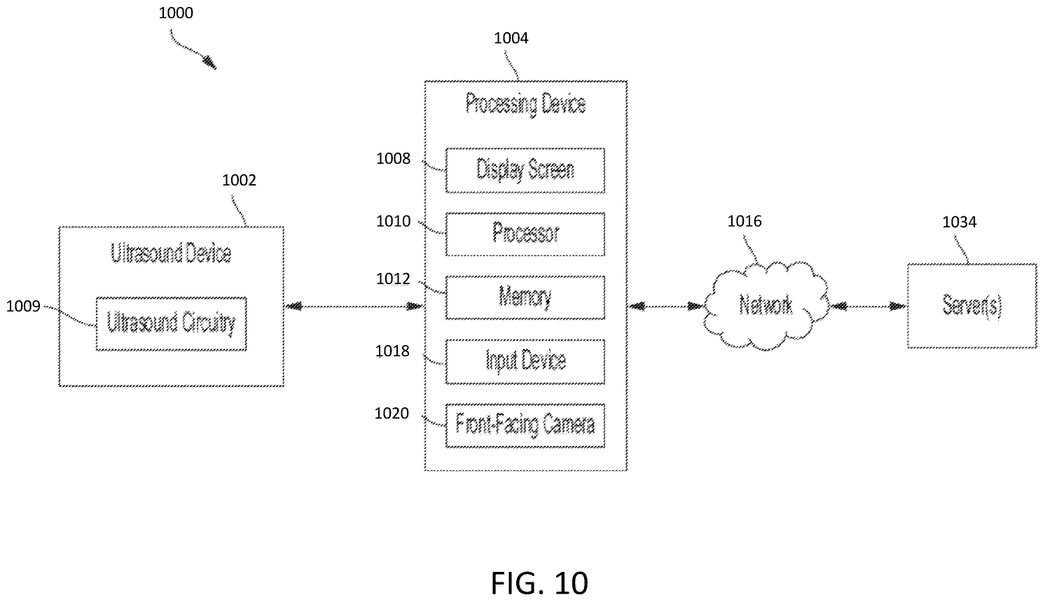

For every focal point that is measured within the field of view (i.e., for every pixel in the final image), the first and second beamformers 116, 118 transmit a first sub-aperture signal 122 and a second sub-aperture signal 124 to a host 130, where the host 130 may include electronic circuitry or a processor that processes the first and second sub-aperture signals 122, 124. In one or more embodiments, the host may be any information processing device such as a smartphone, a tablet, a single-board computer, a laptop computer, or a desktop computer. In these embodiments additional electronic circuitry may be included for digitally sampling the first sub-aperture signal 122 and the second sub-aperture signal 124 and converting these signals into a digital form that can be processed by the above listed devices. Additional details about information processing devices that may be used as the host 130 are provided below, with respect to FIG. 10. However, the host 130 is not limited to a digital information processing device. The host 130 may also be implemented using analog electronic components such as stand-alone electronic components, integrated circuits, and/or circuits implemented within an FPGA.

In one or more embodiments, the host 130 performs a logarithmic detection on both the first sub-aperture signal 122 and the second sub-aperture signal 124 in order to generate a first sub-aperture logarithmic signal 132 and a second sub-aperture logarithmic signal 134. The logarithmic detection of the digitally sampled ultrasound signals generates a signal that is the logarithm of the envelope of the original ultrasound signal waveform. In this way the generated first and second sub-aperture logarithmic signals 132, 134 may be able to produce a final ultrasound image with a higher dynamic range because the upper range of amplitudes of the ultrasound waveform is compressed by the logarithmic detection. The first and second sub-aperture logarithmic signals 132, 134 may also be scaled to a normalization factor that is chosen to achieve an ultrasound image that can be interpreted by a user/operator. In some embodiments, such as those described above where the data processing steps are implemented in electronic circuitry instead of by an information processing system, the logarithmic detection may be implemented by a logarithmic amplifier.

After logarithmic detection, the first sub-aperture logarithmic signal 132 and the second sub-aperture logarithmic signal 134 can be averaged in order to produce an ultrasound image with reduced speckle. Because these two signals 132 and 134 originated from different sub-arrays (104 and 106) that are spatially separated (and therefore the angle of propagation of the corresponding ultrasound waves detected at the first and second sub-apertures 108, 110), the point spread functions of the first and second sub-aperture logarithmic signals 132, 134 are decorrelated. This decorrelation means that the speckle pattern that would be observed in an image generated from only the first sub-aperture logarithmic signal 132 will be different from the speckle pattern that would be observed in an image generated from only the second sub-aperture logarithmic signal 134. Therefore, when the first and second sub-aperture signals 132, 134 are averaged (compounded), the speckle pattern can be significantly reduced.

However, it is important to note that the resolution of an ultrasound image is directly related to the size of the aperture over which the ultrasound waves are detected. Therefore, images generated from only the first sub-aperture logarithmic signal 132 or the second sub-aperture logarithmic signal 134, or indeed, from averaging these two signals 132, 134 must, by definition, have a lower inherent resolution than a similar image that would be generated from the full aperture.

Therefore, in addition to logarithmically detecting the first and second sub-aperture signals 122, 124, in one or more embodiments, the host 130 also coherently adds (i.e., sums) the first and second sub-aperture signals 122, 124 in order to generate a full aperture signal 126. By coherently adding these signals 122 and 124, the phase and amplitudes of the waveforms of the sub-aperture signals 122, 124 are preserved, effectively recreating the signal that would have been detected by the full transducer array (i.e., the full aperture). Because this process effectively re-creates a full aperture signal as it would have been detected via the full array of transducers, there is no degradation in resolution, unlike the lower resolutions resulting from the signals that only use partial or sub-aperture signals. This recreated full aperture signal also includes any constructive or destructive interference (i.e., speckle) that would have been included in a full aperture detection of the ultrasound waves. The host 130 further generates a full-aperture logarithmic signal 136 by also logarithmically detecting the full-aperture signal 126. The host then averages the full-aperture logarithmic signal 136 with the first and second sub-aperture logarithmic signals 132, 134. Because, as mentioned above, there is not degradation of the resolution in the recreated full aperture signal 126, the inclusion of the higher resolution full-aperture logarithmic signal 136 in the average enhances the resolution of the final ultrasound image. In this way, by averaging the three logarithmic signals 132, 134, 136, the host 130 generates an average signal 138 that balances resolution and speckle reduction in order to achieve a high-quality ultrasound image. Additionally, because the full aperture signal is recreated from each of the sub-aperture signals instead of directly detected, the number of beamformers needed to achieve the above-described speckle reduction is minimized to only two in the above-described case (effectively the number of beamforming resources is limited to only the number of sub-apertures employed in receiving the signals. Furthermore, because the first and second sub-arrays 104, 106 detect the ultrasound waves in parallel, the first and second beamformers 116, 118 may beamform the signals in parallel, and the processing of the different signals within the host 130 may also be performed in parallel, a high frame rate can be achieved by the above-described aperture compounding ultrasound system 100.

In one or more embodiments, the above-described generation of the average signal may be iteratively repeated in order to generate an ultrasound image of the average signals 138 at every focal point (i.e., pixel). This may include iteratively repeating both the beamforming performed in the beamformers 116, 118 and/or the processing performed in the host 130. In other embodiments, the beamforming and/or the processing by the host 130 may be performed in parallel for multiple focal points (i.e., pixels) in order to generate a final ultrasound image.

In the above description, it is assumed that the generated average signal corresponds to only one transmit event. However, in some embodiments, there may also be separate transmit events from each of the sub-apertures and/or from the full aperture that are combined to generate an average signal for each particular focal point/pixel. The first sub-array 104 of transducers may transmit, from the first sub-aperture 108, a transmitted first sub-aperture signal, and the second sub-array 106 of transducers may transmit, from the second sub-aperture 110, a transmitted second sub-aperture signal. These transmissions may be separate transmission events, for which ultrasound waves, that are scattered or reflected by the medium being imaged, are received and detected at both the first sub-aperture and the second sub-aperture.

Once the ultrasound waves are received/detected for each transmit event, the above-described process of beamforming the signals received by both the first sub-aperture 108 and the second sub-aperture 110 is repeated for each received sub-aperture signal. The beamforming process generates a first sub-aperture signal 122 and a second sub-aperture signal 124 corresponding to each of the transmitted first sub-aperture signal and the transmitted second sub-aperture signal. Each of these signals can then be processed by the host 130. As described above, the host coherently adds the first sub-aperture signal 122 and the second sub-aperture signal 124 to generate a full-aperture signal 126 associated with each of the transmitted first sub-aperture signal and the transmitted second sub-aperture signal. Further, for each of the transmitted sub-aperture signals, the host 130 logarithmically detects the first sub-aperture signal 122, the second sub-aperture signal 124 and the full aperture signal 126, as described above.

Additionally, in some embodiments, a full-array transmission event may also be included in addition to the first sub-aperture transmission and the second sub-aperture transmission described above. In this case, the full array of transducers (i.e., the first sub-array 104 and the second sub-array 106 together) physically transmit, from the full aperture, a transmitted full aperture signal. Ultrasound waves corresponding to the transmitted full aperture signal are then scattered by the medium and received/detected at the first sub-aperture 108 and the second sub-aperture 110. Signals corresponding to these ultrasound waves detected at the first sub-aperture 108 and the second sub-aperture 110 are then beamformed by the beamformers 116, 118, and then processed by the host 130, as described above, resulting in three logarithmically detected signals corresponding to the first sub-aperture, second sub-aperture and the full aperture.

However, in other embodiments, a transmitted full aperture signal can be recreated from the transmitted first sub-aperture signal and the transmitted second sub-aperture signal. In this case, a full-aperture signal may not be physically transmitted by the transducer array. Instead, similar to the coherent addition of multiple received sub-aperture signals as described above, the host 130 may coherently sum the transmitted first sub-aperture signals and the transmitted second sub-aperture signal in order to synthesize, or recreate, a received signal that would have resulted from a physically transmitted full aperture signal. In some embodiments, this may be achieved by processing different combinations of beamformed signals by the host 130. For example, in order to achieve a first sub-aperture signal 122 that corresponds to the synthesized or recreated transmitted full aperture signal, the first sub-aperture signal 122 corresponding to the transmit event from the first sub-aperture 108 (i.e., corresponding to the transmitted first sub-aperture signal) and the first sub-aperture signal 122 corresponding to the transmit event from the second sub-aperture 110 (i.e., corresponding to the transmitted second sub-aperture signal) may be coherently added by the host 130. Similarly, in order to achieve a second sub-aperture signal 124 that corresponds to the synthesized or recreated transmitted full aperture signal, the second sub-aperture signal 124 corresponding to the transmit event from the first sub-aperture 108 (i.e., corresponding to the transmitted first sub-aperture signal) and the second sub-aperture signal 124 corresponding to the transmit event from the second sub-aperture 110 (i.e., corresponding to the transmitted second sub-aperture signal) may be coherently added by the host 130. Finally, in order to achieve a full aperture signal 126 that corresponds to the synthesized or recreated transmitted full aperture signal, both first sub-aperture signals 122 from both transmit events and both second sub-aperture signals 124 from both transmit events may all be coherently added together.

In this way, as described above, nine possible received and/or recreated signals may exist for each pixel of each ultrasound image/frame. For clarity, the nine signals can be represented as follows where the first identifier corresponds to the transmission event from which the signal originates, and the second identifier corresponds to the aperture (physical or recreated) through which the signal is received (i.e., transmitted aperture/received aperture). The nine signals are therefore: First/First, First/Second, First/Full, Second/First, Second/Second, Second/Full, Full/First, Full/Second, Full/Full. All of these nine signals may be averaged together, or each of the nine signals may then be averaged together, by the host 130, in various different combinations in order to achieve speckle reduction in ultrasound images.

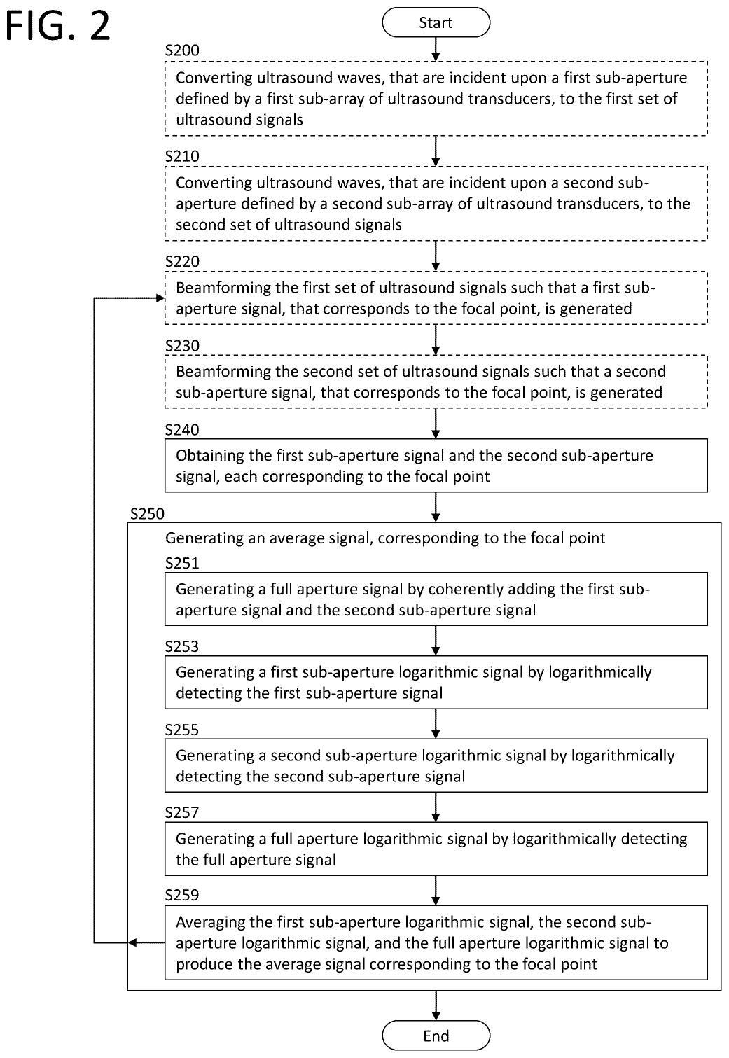

FIG. 2 shows a flow chart for a method for aperture compounding in ultrasound imaging, according to one or more embodiments. One or more individual processes shown in FIG. 2 may be omitted, repeated, and/or performed in a different order than the order shown in FIG. 2. Accordingly, the scope of the invention should not be limited by the specific arrangement as depicted in FIG. 2. Additionally, according to one or more embodiments, the method depicted in FIG. 2 (and described below) may be implemented using the above-described ultrasound system 100 as well as any variation of the ultrasound system 100 or any other suitable system or apparatus. Some or all of the steps in the method depicted in FIG. 2 may be performed by a processor, and the instructions for performing these steps may be stored in a non-transitory computer readable memory.

At S200, ultrasound waves that are incident upon the first sub-aperture 108, defined by the first sub-array 104 of ultrasound transducers, are converted to the first set of ultrasound signals 112.

At S210, ultrasound waves that are incident upon the second sub-aperture 110, defined by the second sub-array 106 of ultrasound transducers, are converted to the second set of ultrasound signals 114.

At S220, the first set of ultrasound signals 112 are beamformed to generate a first sub-aperture signal 122 that corresponds to the focal point (i.e., image pixel).

At S230, the second set of ultrasound signals 114 are beamformed to generate a second sub-aperture signal 124 that corresponds to the focal point (i.e., image pixel). In one or more embodiments, S200-S230 are optional and may be omitted from the method.

At S240, the first sub-aperture signal 122 and the second sub-aperture signal 124, each corresponding to the focal point, are obtained for processing.

At S250, the average signal 138 is generated. S250 further comprises S251, S253, S255, S257, S259, which are described below, and result in the generation of the average signal 138.

At S251, the full aperture signal 126 is generated by coherently adding the first sub-aperture signal 122 and the second sub-aperture signal 124.

At S253, the first sub-aperture logarithmic signal 132 is generated by logarithmically detecting the first sub-aperture signal 122.

At S255, the second sub-aperture logarithmic signal 134 is generated by logarithmically detecting the second sub-aperture signal 124.

At S257, the full-aperture logarithmic signal 136 is generated by logarithmically detecting the full-aperture signal 126.

At S259, the first sub-aperture logarithmic signal 132, the second sub-aperture logarithmic signal 134, and the full aperture logarithmic signal 136 are averaged to produce the average signal 138 corresponding to the focal point.

In this way, as also discussed above, by averaging the signals 132, 134, and 136 (i.e., aperture compounding), an ultrasound image with reduced speckle can be generated while maintaining a high image resolution.

Additionally, in one or more embodiments, steps S220-S250 may be iteratively repeated at multiple different focal points within the medium in order to generate ultrasound image data at each pixel within the image. In other embodiments, these steps may be performed for multiple focal points (i.e., pixels) in parallel.

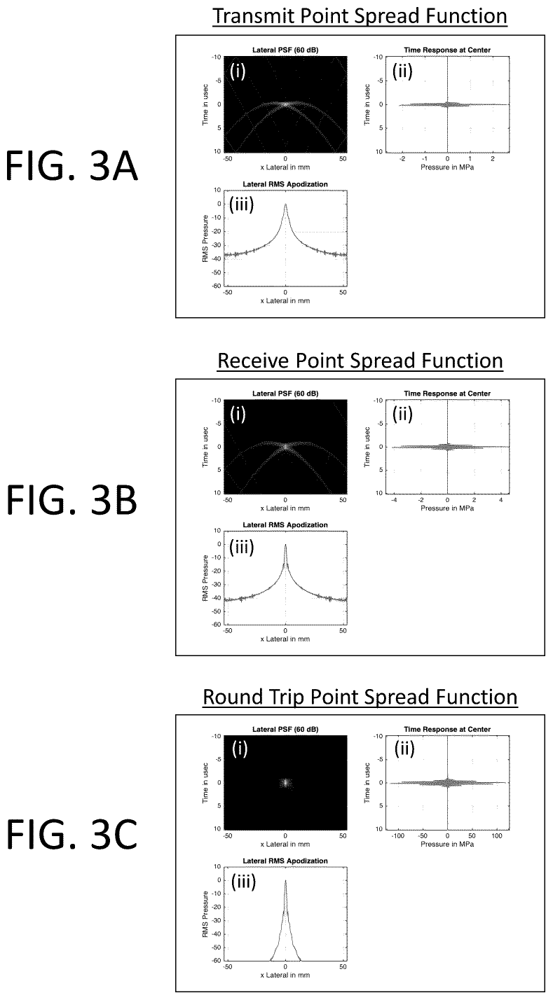

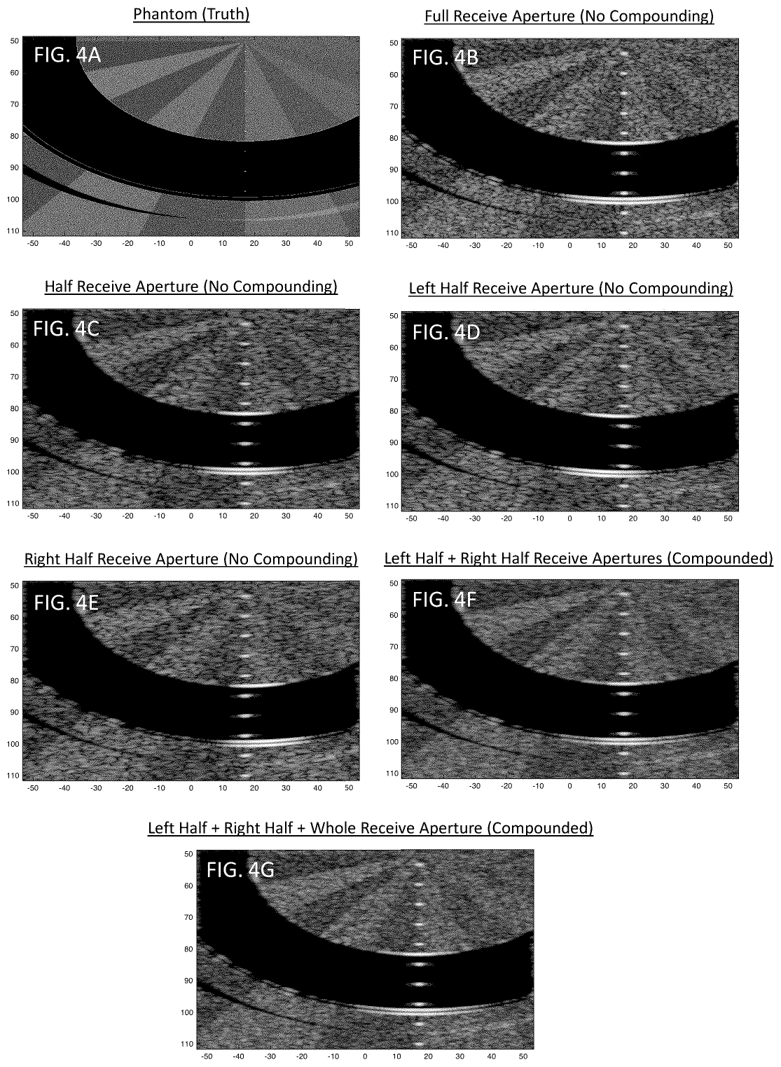

Turning now to FIGS. 3A-3C and 4A-4G, simulations illustrating speckle reduction by aperture compounding, according to one or more embodiments, will be described. In the simulations of FIGS. 3A-3C and 4A-4G described below, a computer-generated phantom was employed that includes both specular reflectors and Rayleigh scatters. The simulation assumes that the PSF is space-invariant over a small region of space, and the PSF is calculated at the observation depth of 80 millimeters. Additionally, the model is two-dimensional in that it only includes the azimuthal (lateral) and depth dimensions, and the elevation dimension is excluded. For the simulated transmitted signal, a frequency of 3 MHZ, a 70% fractional bandwidth, a 20 millimeter aperture, and a 100 millimeter focus depth were used. The received signal was modeled with a 3 MHz frequency, a 50% fractional bandwidth, a full aperture size of 30 millimeters, and an 80 millimeter focus depth. In order to achieve aperture compounding, the full 30 millimeter aperture was divided into two 15 millimeter apertures (Left and Right) that are spatially separated in the lateral direction of the transducer array.

FIGS. 3A-3C show simulated point spread functions (PSFs) for transmission (FIG. 3A), reception (FIG. 3B), and for a round trip of the ultrasound waves (FIG. 3C, i.e. transmission and reception), according to one or more embodiments. For each of FIGS. 3A-3C, subplot (i) shows a two-dimensional plot of the lateral PSF, where time (in microseconds) is the vertical axis and lateral position (in mm) is the horizontal axis. Additionally, subplot (ii) shows the time response at the center (i.e., the slice of subplot (i) along the line where the lateral position is zero. Subplot (iii) shows the lateral root mean square (RMS) apodization (i.e., the slice through subplot (i) along the line where the time equals zero.

FIGS. 4A-4G show simulations of ultrasound images that may be acquired in various modes of operation of the ultrasound system 100, according to one or more embodiments. FIG. 4A shows the physical structure of the simulated sample as it was actually specified in the simulations (in other words, FIG. 4A does not show a simulated ultrasound image, but rather the inherent structure of the simulated sample). FIG. 4B shows a simulated conventional full aperture ultrasound image without any compounding. In this case, a high resolution is achieved, but the speckle pattern in the image (i.e., randomly oriented bright and dark areas in regions that should have constant contrast as shown in FIG. 4A) is prominent. FIGS. 4C, 4D, and 4E each show simulated images taken using half of the full aperture. For example, FIGS. 4D and 4E show simulated images taken via the left half receive aperture and the right half receive aperture (i.e., corresponding to the first and second sub-apertures 108, 110 in the above description of the ultrasound system 100 above). In each of these cases, there is no compounding, and therefore the speckle pattern is still prominent. However, in each of these cases the resolution of the image is somewhat lower because a smaller receive aperture is being used. Additionally, a close comparison of FIGS. 4D and 4E reveals that the speckle patterns are different because the PSFs of these images are decorrelated as described above. FIG. 4F shows the case where the left half receive aperture is compounded with the right half receive aperture (i.e., FIGS. 4D and 4E are averaged). Because the speckle pattern is decorrelated, averaging of these two leads to significantly reduced speckle. FIG. 4G shows the case where the left half, right half, and whole apertures are compounded. In comparison with FIG. 4F, FIG. 4G maintains the reduced speckle noise but also has a higher resolution due to the compounding of the half aperture images with the full aperture image.

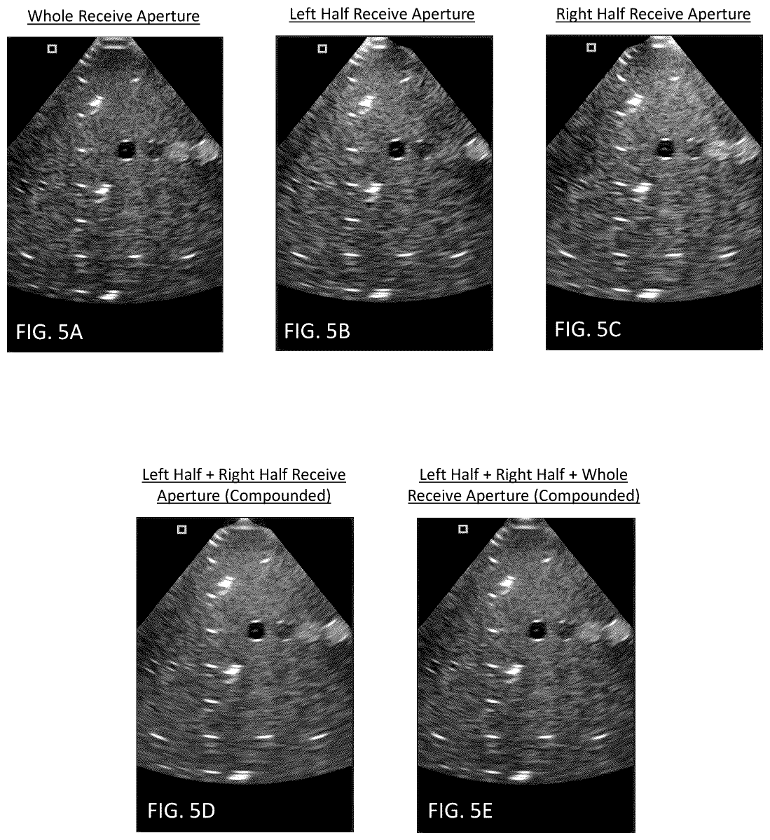

FIGS. 5A-5E show ultrasound images acquired using the ultrasound system 100, according to one or more embodiments. FIG. 5A shows a conventional ultrasound image acquired using the whole receive aperture (i.e., the full aperture). FIG. 5B and FIG. 5C show ultrasound images acquired using only the left half receive aperture and the right half receive aperture, respectively. Again, the speckle pattern is different between these two images because the PSF is decorrelated. Therefore, as shown in FIG. 5D, the left half aperture signal and the right half aperture signal can be compounded to reduce the prominence of the speckle pattern. FIG. 5E shows an ultrasound image where the left half receive aperture, the right half receive aperture, and the whole aperture are compounded to generate an ultrasound image that has reduced speckle with respect to FIG. 5A, but also has a higher resolution than images based only on half aperture signals.

FIG. 6 shows a block diagram of an ultrasound system 100 for reducing speckle in ultrasound data by aperture compounding, according to one or more embodiments. FIG. 6 is similar to FIG. 1, except that the first and second sub-arrays 104, 106 are spatially intermittent in the lateral direction because the first and second sub-arrays 104, 106 each include non-consecutive groups 105, 107 of the ultrasound transducers in the array of ultrasound transducers. In some embodiments, these nonconsecutive group of the first and second sub-arrays 105, 107 may also be interleaved such that the spatially intermittent first and second sub-apertures overlap in the lateral direction. In other words, each of the nonconsecutive groups of transducers in the first sub-array 105 may be directly adjacent to the nonconsecutive groups of transducers of the second sub-array 107, as shown in FIG. 6. In this way, compared to the embodiment shown in FIG. 1, a higher resolution may be achieved while maintaining the ability to reduce speckle because both the first and second sub-apertures 108, 110 are effectively larger (end to end in the lateral direction), as shown in FIG. 6. However, this approach may also result in grating lobes and/or modulation of the PSF. As shown in FIG. 6, the first and second sub-apertures 108, 110 are each divided into two non-consecutive chunks (i.e., the non-consecutive groups of transducers 105, 107). However, in other embodiments each sub-aperture 108, 110 may be divided into any number of suitable non-consecutive chunks.

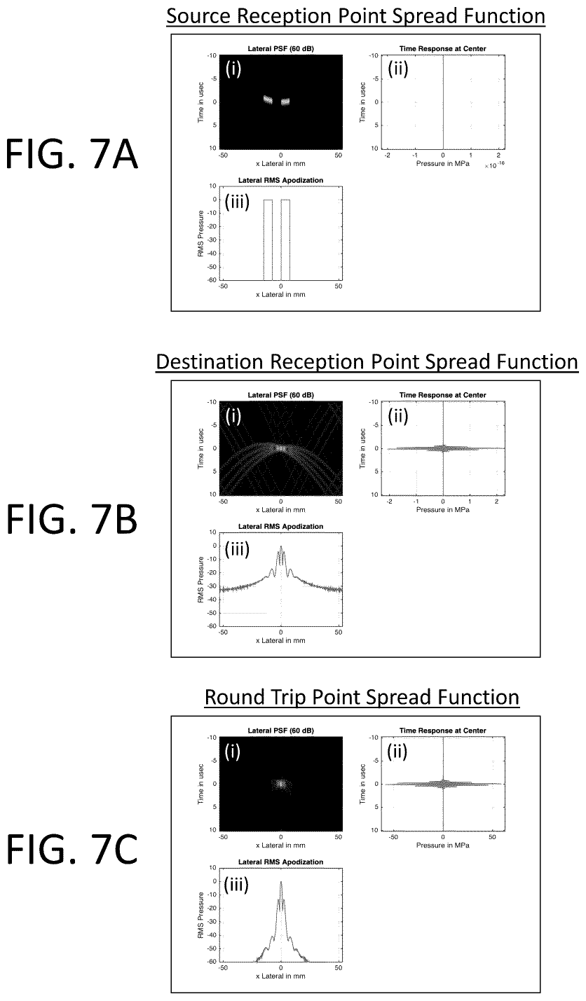

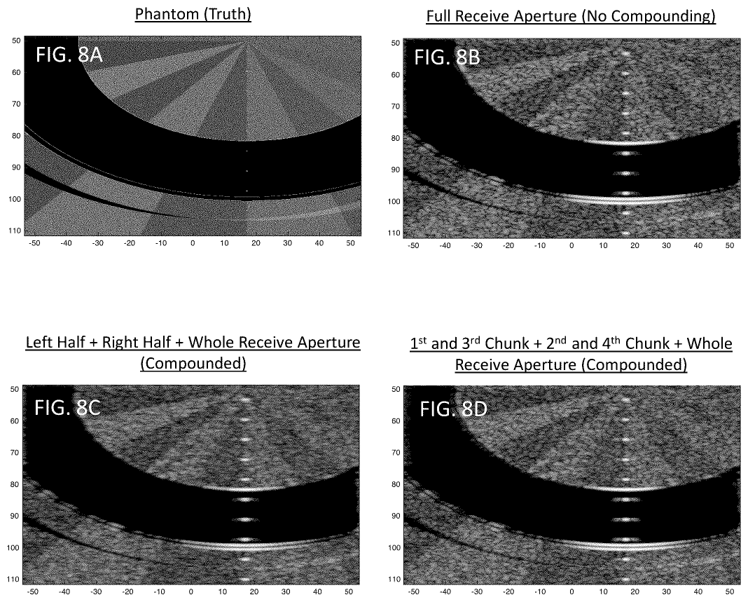

Turning now to FIGS. 7A-7C and 8A-8D, simulations illustrating speckle reduction by aperture compounding, according to one or more embodiments, will be described. The simulations of the FIGS. 7 and 8, use the same parameters described above with respect to the simulations of FIGS. 3 and 4, except that the 30 millimeter full aperture is divided into four 7.5 millimeter chunks, where the first and third chunks belong to the first sub-aperture 108 and the second and fourth chunks belong to the second sub-aperture 110. Therefore, the first and second sub-apertures 108, 110 are effectively 22.5 millimeters, which is larger than the embodiment of the ultrasound system 100 used in the simulations shown in FIGS. 3 and 4.

FIGS. 7A-7C show simulated point spread functions (PSFs) for reception at the source (FIG. 7A) and at the destination (i.e., focus, FIG. 7B), and for a round trip of the ultrasound waves (FIG. 7C, i.e. transmission and reception), according to one or more embodiments. Specifically, the PSFs shown correspond to the first and third chunks (i.e., one of the two sub-apertures). For each of FIGS. 7A-7C, subplot (i) shows a two-dimensional plot of the lateral PSF, where time (in microseconds) is the vertical axis and lateral position (in mm) is the horizontal axis. Additionally, subplot (ii) shows the time response at the center (i.e., the slice of subplot (i) along the line where the lateral position is zero. Subplot (iii) shows the lateral root mean square (RMS) apodization (i.e., the slice through subplot (i) along the line where the time equals zero.

Combining the two spatially separated chunks results in a sinusoidal modulation of the destination PSF (FIG. 7B). However, convolving with the transmission PSF can mitigate this effect, but cannot completely eliminate the sinusoidal modulation, as seen in the round trip PSF in FIG. 7C.

FIGS. 8A-8D show simulations of ultrasound images that may be acquired in various modes of operation of the ultrasound system 100, according to one or more embodiments. FIGS. 8A, 8B, and 8C are equivalent reproductions of FIGS. 4A, 4B, and 4G, respectfully, and are included only for comparison with FIG. 8D. FIG. 8D shows the ultrasound image where the first and third aperture chunks are compounded with the second and fourth aperture chunks, and these are also compounded with the whole aperture. In comparison with FIG. 8C, FIG. 8D shows improved resolution because the effective size of the two sub-apertures is larger.

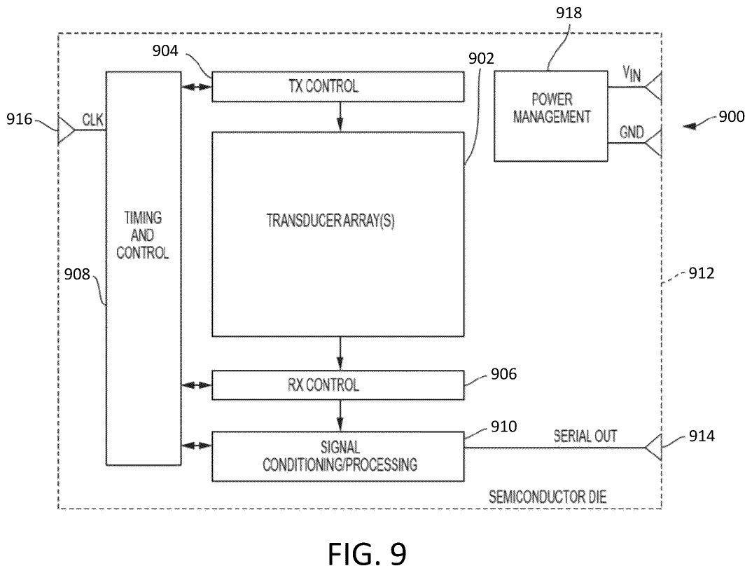

FIG. 9 is a block diagram of an example of an ultrasound device in accordance with some embodiments of the technology described herein. The illustrated ultrasound device 900 may be included in the above-described ultrasound system 100 and may implement the signal processing techniques described herein, including the coherence imaging techniques described herein. The illustrated ultrasound device 900 may include one or more ultrasonic transducer arrangements (e.g., arrays) 902, transmit (TX) circuitry 904, receive (RX) circuitry 906, a timing and control circuit 908, a signal conditioning/processing circuit 910, and/or a power management circuit 918.

The one or more ultrasonic transducer arrays 902 may take on any of numerous forms, and aspects of the present technology do not necessarily require the use of any particular type or arrangement of ultrasonic transducer cells or ultrasonic transducer elements. For example, multiple ultrasonic transducer elements in the ultrasonic transducer array 902 may be arranged in one-dimension, or two-dimensions. Although the term “array” is used in this description, it should be appreciated that in some embodiments the ultrasonic transducer elements may be organized in a non-array fashion. In various embodiments, each of the ultrasonic transducer elements in the array 902 may, for example, include one or more capacitive micromachined ultrasonic transducers (CMUTs), or one or more piezoelectric micromachined ultrasonic transducers (PMUTs).

In a non-limiting example, the ultrasonic transducer array 902 may include between approximately 6,000-10,000 (e.g., 8,960) active CMUTs on the chip, forming an array of hundreds of CMUTs by tens of CMUTs (e.g., 140×64). The CMUT element pitch may be between 150-250 um, such as 208 um, and thus, result in the total dimension of between 10-50 mm by 10-50 mm (e.g., 29.12 mm×13.312 mm).

In some embodiments, the TX circuitry 904 may, for example, generate pulses that drive the individual elements of, or one or more groups of elements within, the ultrasonic transducer array(s) 902 so as to generate acoustic signals to be used for imaging. The RX circuitry 906, on the other hand, may receive and process electronic signals generated by the individual elements of the ultrasonic transducer array(s) 902 when acoustic signals impinge upon such elements.

With further reference to FIG. 9, in some embodiments, the timing and control circuit 908 may be, for example, responsible for generating all timing and control signals that are used to synchronize and coordinate the operation of the other elements in the device 900. In the example shown, the timing and control circuit 908 is driven by a single clock signal CLK supplied to an input port 916. The clock signal CLK may be, for example, a high-frequency clock used to drive one or more of the on-chip circuit components. In some embodiments, the clock signal CLK may, for example, be a 1.5625 GHz or 2.5 GHz clock used to drive a high-speed serial output device (not shown in FIG. 9) in the signal conditioning/processing circuit 910, or a 20 Mhz or 40 MHz clock used to drive other digital components on the die 912, and the timing and control circuit 908 may divide or multiply the clock CLK, as necessary, to drive other components on the die 912. In other embodiments, two or more clocks of different frequencies (such as those referenced above) may be separately supplied to the timing and control circuit 908 from an off-chip source.

In some embodiments, the output range of a same (or single) transducer unit in an ultrasound device may be anywhere in a range of 1-12 MHz (including the entire frequency range from 1-12 MHz), making it a universal solution, in which there is no need to change the ultrasound heads or units for different operating ranges or to image at different depths within a patient. That is, the transmit and/or receive frequency of the transducers of the ultrasonic transducer array may be selected to be any frequency or range of frequencies within the range of 1 MHZ-12 MHz. The universal device 900 described herein may thus be used for a broad range of medical imaging tasks including, but not limited to, imaging a patient's liver, kidney, heart, bladder, thyroid, carotid artery, lower venous extremity, and performing central line placement. Multiple conventional ultrasound probes would have to be used to perform all these imaging tasks. By contrast, a single universal ultrasound device 900 may be used to perform all these tasks by operating, for each task, at a frequency range appropriate for the task, as shown in the examples of Table 1 together with corresponding depths at which the subject may be imaged.

TABLE 1
Illustrative depths and frequencies at which an ultrasound
device implemented in accordance with embodiments
described herein may image a subject.

| | | |
|---|---|---|
| Organ | Frequencies | Depth (up to) |

| | | | | |
|---|---|---|---|---|
| Liver/Right Kidney | 2-5 | MHz | 15-20 | cm |
| Cardiac (adult) | 1-5 | MHz | 20 | cm |

| | | |
|---|---|---|
| Bladder | 2-5 MHz; 3-6 MHz | 10-15 cm; 5-10 cm |

| | | | | |
|---|---|---|---|---|
| Lower extremity | 4-7 | MHz | 4-6 | cm |
| venous | | | | |
| Thyroid | 7-12 | MHz | 4 | cm |
| Carotid | 5-10 | MHz | 4 | cm |
| Central Line | 5-10 | MHz | 4 | cm |
| Placement | | | | |

The power management circuit 918 may be, for example, responsible for converting one or more input voltages VIN from an off-chip source into voltages needed to carry out operation of the chip, and for otherwise managing power consumption within the device 900. In some embodiments, for example, a single voltage (e.g., 12V, 80V, 100V, 120V, etc.) may be supplied to the chip and the power management circuit 918 may step that voltage up or down, as necessary, using a charge pump circuit or via some other DC-to-DC voltage conversion mechanism. In other embodiments, multiple different voltages may be supplied separately to the power management circuit 918 for processing and/or distribution to the other on-chip components.

In the embodiment shown above, all of the illustrated elements are formed on a single semiconductor die 912. It should be appreciated, however, that in alternative embodiments one or more of the illustrated elements may be instead located off-chip, in a separate semiconductor die, or in a separate device. Alternatively, one or more of these components may be implemented in a DSP chip, a field programmable gate array (FPGA) in a separate chip, or a separate application specific integrated circuitry (ASIC) chip. Additionally, and/or alternatively, one or more of the components in the beamformer may be implemented in the semiconductor die 912, whereas other components in the beamformer may be implemented in an external processing device in hardware or software, where the external processing device is capable of communicating with the ultrasound device 900.

In addition, although the illustrated example shows both TX circuitry 904 and RX circuitry 906, in alternative embodiments only TX circuitry or only RX circuitry may be employed. For example, such embodiments may be employed in a circumstance where one or more transmission-only devices are used to transmit acoustic signals and one or more reception-only devices are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

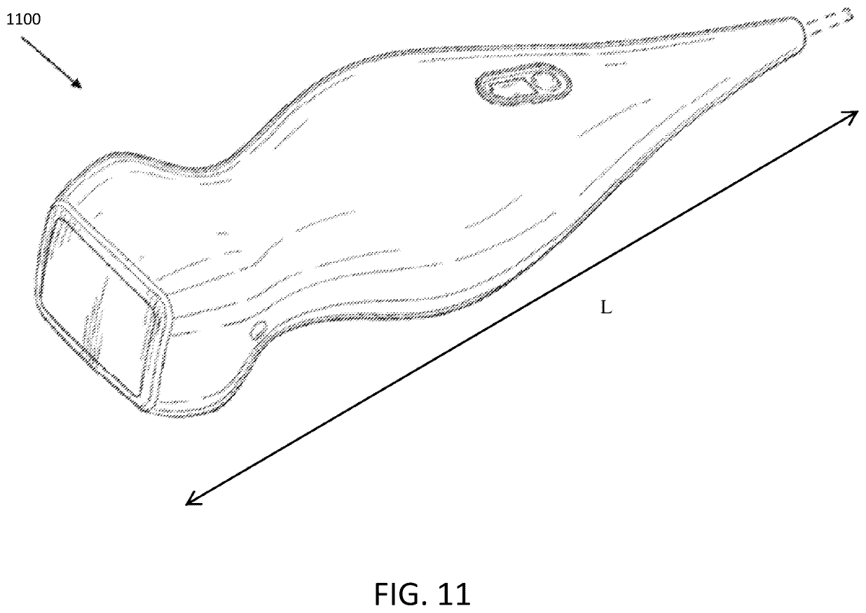

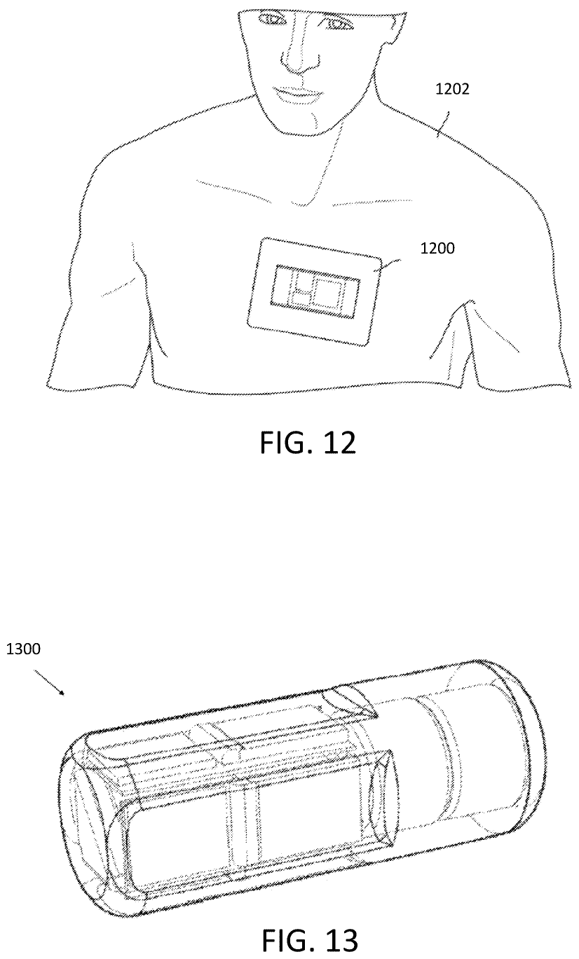

In some embodiments, the ultrasonic transducer elements of the ultrasonic transducer array 902 may be formed on the same chip as the electronics of the TX circuitry 904 and/or RX circuitry 906. The ultrasonic transducer arrays 902, TX circuitry 904, and RX circuitry 906 may be, in some embodiments, integrated in a single ultrasound probe. In some embodiments, the single ultrasound probe may be a hand-held probe including, but not limited to, the hand-held probes described below with reference to FIG. 11. In other embodiments, the single ultrasound probe may be embodied in a patch that may be coupled to a patient. FIG. 12 provides a non-limiting illustration of such a patch. The patch may be configured to transmit, wirelessly, data collected by the patch to one or more external devices for further processing. In other embodiments, the single ultrasound probe may be embodied in a pill that may be swallowed by a patient. The pill may be configured to transmit, wirelessly, data collected by the ultrasound probe within the pill to one or more external devices for further processing. FIG. 13 illustrates a non-limiting example of such a pill.

A CMUT may include, for example, a cavity formed in a CMOS wafer, with a membrane overlying the cavity, and in some embodiments sealing the cavity. Electrodes may be provided to create an ultrasonic transducer cell from the covered cavity structure. The CMOS wafer may include integrated circuitry to which the ultrasonic transducer cell may be connected. The ultrasonic transducer cell and CMOS wafer may be monolithically integrated, thus forming an integrated ultrasonic transducer cell and integrated circuit on a single substrate (the CMOS wafer).

In the example shown, one or more output ports 914 may output a high-speed serial data stream generated by one or more components of the signal conditioning/processing circuit 910. Such data streams may be, for example, generated by one or more USB 3.0 modules, and/or one or more 10 GB, 40 GB, or 100 GB Ethernet modules, integrated on the die 912. It is appreciated that other communication protocols may be used for the output ports 914.

In some embodiments, the signal stream produced on output port 914 can be provided to a computer, tablet, or smartphone for the generation and/or display of two-dimensional, three-dimensional, and/or tomographic images. In some embodiments, the signal provided at the output port 914 may be ultrasound data provided by the one or more beamformer components or auto-correlation approximation circuitry 928, where the ultrasound data may be used by the computer (external to the ultrasound device) for displaying the ultrasound images. In embodiments in which image formation capabilities are incorporated in the signal conditioning/processing circuit 910, even relatively low-power devices, such as smartphones or tablets which have only a limited amount of processing power and memory available for application execution, can display images using only a serial data stream from the output port 914. As noted above, the use of on-chip analog-to-digital conversion and a high-speed serial data link to offload a digital data stream is one of the features that helps facilitate an “ultrasound on a chip” solution according to some embodiments of the technology described herein.

Devices 900 such as that shown in FIG. 9 may be used in various imaging and/or treatment (e.g., HIFU) applications, and the particular examples described herein should not be viewed as limiting. In one illustrative implementation, for example, an imaging device including an N×M planar or substantially planar array of CMUT elements may itself be used to acquire an ultrasound image of a subject (e.g., a person's abdomen) by energizing some or all of the elements in the ultrasonic transducer array(s) 902 (either together or individually) during one or more transmit phases, and receiving and processing signals generated by some or all of the elements in the ultrasonic transducer array(s) 902 during one or more receive phases, such that during each receive phase the CMUT elements sense acoustic signals reflected by the subject. In other implementations, some of the elements in the ultrasonic transducer array(s) 902 may be used only to transmit acoustic signals and other elements in the same ultrasonic transducer array(s) 902 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device 900 or on a single die 912.

FIG. 10 illustrates a schematic block diagram of an example ultrasound system 1000 which may implement various aspects of the technology described herein. In some embodiments, ultrasound system 1000 may include an ultrasound device 1002, an example of which is implemented in ultrasound device 900. For example, the ultrasound device 1002 may be a handheld ultrasound probe. Additionally, the ultrasound system 1000 may include a processing device 1004 (for example, the host 130, as described above), a communication network 1016, and one or more servers 1034. The ultrasound device 1002 may be configured to generate ultrasound data that may be employed to generate an ultrasound image. The ultrasound device 1002 may be constructed in any of a variety of ways. In some embodiments, the ultrasound device 1002 includes a transmitter that transmits a signal to a transmit beamformer which in turn drives transducer elements within a transducer array to emit pulsed ultrasound signals into a structure, such as a patient. The pulsed ultrasound signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. In some embodiments, the ultrasound device 1002 may include an ultrasound circuitry 1009 that may be configured to generate the ultrasound data. For example, the ultrasound device 1002 may include semiconductor die 912 for implementing the various techniques described in.

Reference is now made to the processing device 1004. In some embodiments, the processing device 1004 may be communicatively coupled to the ultrasound device 1002 (e.g., 900 in FIG. 9) wirelessly or in a wired fashion (e.g., by a detachable cord or cable) to implement at least a portion of the process for approximating the auto-correlation of ultrasound signals. For example, one or more beamformer components (of FIG. 9) may be implemented on the processing device 1004. In some embodiments, the processing device 1004 may include one or more processing devices (processors) 1010, which may include specially-programmed and/or special-purpose hardware such as an ASIC chip. The processor 1010 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

In some embodiments, the processing device 1004 may be configured to process the ultrasound data received from the ultrasound device 1002 to generate ultrasound images for display on the display screen 1008. The processing may be performed by, for example, the processor(s) 1010. The processor(s) 1010 may also be adapted to control the acquisition of ultrasound data with the ultrasound device 1002. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

In some embodiments, the processing device 1004 may be configured to perform various ultrasound operations using the processor(s) 1010 (e.g., one or more computer hardware processors) and one or more articles of manufacture that include non-transitory computer-readable storage media such as the memory 1012. The processor(s) 1010 may control writing data to and reading data from the memory 1012 in any suitable manner. To perform certain of the processes described herein, the processor(s) 1010 may execute one or more processor-executable instructions stored in one or more non-transitory computer-readable storage media (e.g., the memory 1012), which may serve as non-transitory computer-readable storage media storing processor-executable instructions for execution by the processor(s) 1010.

The camera 1020 may be configured to detect light (e.g., visible light) to form an image. The camera 1020 may be on the same face of the processing device 1004 as the display screen 1008. The display screen 1008 may be configured to display images and/or videos, and may be, for example, a liquid crystal display (LCD), a plasma display, and/or an organic light emitting diode (OLED) display on the processing device 1004. The input device 1018 may include one or more devices capable of receiving input from a user and transmitting the input to the processor(s) 1010. For example, the input device 1018 may include a keyboard, a mouse, a microphone, touch-enabled sensors on the display screen 1008, and/or a microphone. The display screen 1008, the input device 1018, the camera 1020, and/or other input/output interfaces (e.g., speaker) may be communicatively coupled to the processor(s) 1010 and/or under the control of the processor 1010.

It should be appreciated that the processing device 1004 may be implemented in any of a variety of ways. For example, the processing device 1004 may be implemented as a handheld device such as a mobile smartphone or a tablet. Thereby, a user of the ultrasound device 1002 may be able to operate the ultrasound device 1002 with one hand and hold the processing device 1004 with another hand. In other examples, the processing device 1004 may be implemented as a portable device that is not a handheld device, such as a laptop. In yet other examples, the processing device 1004 may be implemented as a stationary device such as a desktop computer. The processing device 1004 may be connected to the network 1016 over a wired connection (e.g., via an Ethernet cable) and/or a wireless connection (e.g., over a WiFi network). The processing device 1004 may thereby communicate with (e.g., transmit data to or receive data from) the one or more servers 1034 over the network 1016. For example, a party may provide from the server 1034 to the processing device 1004 processor-executable instructions for storing in one or more non-transitory computer-readable storage media (e.g., the memory 1012) which, when executed, may cause the processing device 1004 to perform ultrasound processes. FIG. 10 should be understood to be non-limiting. For example, the ultrasound system 1000 may include fewer or more components than shown and the processing device 1004 and ultrasound device 1002 may include fewer or more components than shown. In some embodiments, the processing device 1004 may be part of the ultrasound device 1002.

FIG. 11 illustrates an example handheld ultrasound probe, in accordance with certain embodiments described herein. Handheld ultrasound probe 1100 may implement the ultrasound device 900 in FIG. 9. The handheld ultrasound probe 1100 may be an ultrasound device, e.g., 900 (FIG. 9) or 1002 (FIG. 10) in operative communicative with a processing device (e.g., 1004) and transmit the detected signals to the processing device. Alternatively and/or additionally, the ultrasound probe 1100 may include an ultrasound device and a processing device for performing operations over ultrasound signals received from the ultrasonic transducer. In some embodiments, the handheld ultrasound probe 1100 may be configured to communicate with the processing device (e.g., 1004) wired or wirelessly. Thus, the handheld ultrasound probe 1100 may have a suitable dimension and weight. For example, the ultrasound probe 1100 may have a cable for wired communication with a processing device, and have a length L about 100 mm-300 mm (e.g., 175 mm) and a weight about 200 grams-500 grams (e.g., 312 g). In another example, the ultrasound probe 1100 may be capable of communicating with a processing device wirelessly. As such, the handheld ultrasound probe 1100 may have a length about 140 mm and a weight about 265 g. It is appreciated that other dimensions and weight may be possible.

FIG. 12 illustrates an example wearable ultrasound patch, in accordance with certain embodiments described herein. The wearable ultrasound patch 1200 is coupled to a subject 1202. The wearable ultrasound patch 1200 may be the same as the ultrasound device 900 (FIG. 9) or 1002 (in FIG. 10).

FIG. 13 illustrates an example ingestible ultrasound pill, in accordance with certain embodiments described herein. The ingestible ultrasound pill 1300 may be the same as the ultrasound device 900 (FIG. 9) or 1002 (FIG. 10).

Further description of ultrasound devices and systems may be found in U.S. Pat. No. 9,521,991, the content of which is incorporated by reference herein in its entirety; and U.S. Pat. No. 11,311,274, the content of which is incorporated by reference herein in its entirety.

One or more embodiments of the disclosure may have one or more of the following advantages and improvements over conventional ultrasound imaging systems and ultrasound imaging methods: reduction of speckle noise in ultrasound images; improved resolution in reduced-speckle ultrasound images; improved contrast in reduced-speckle ultrasound images; a faster framerate for generating reduced-speckle ultrasound images. Furthermore, each of the above-listed advantages of embodiments of the disclosure may additionally result in: improved interpretation of ultrasound images for diagnostic and therapeutic applications; improved efficiency in ultrasound-based diagnosis and therapy.

Although the disclosure has been described with respect to a limited number of embodiments, those skilled in the art, having benefit of this disclosure, will appreciate that various other embodiments may be devised without departing from the scope of the present disclosure. Accordingly, the scope of the disclosure should be limited only by the attached claims.

What is claimed is:

1. An aperture compounding method for reducing speckle in ultrasound data corresponding to ultrasound waves received by an array of ultrasound transducers, distributed in a lateral direction, the method comprising:
obtaining a beamformed first sub-aperture signal and a beamformed second sub-aperture signal, each corresponding to a focal point, wherein
the first sub-aperture signal corresponds to a first sub-aperture defined by a first sub-array of the ultrasound transducers, and
the second sub-aperture signal corresponds to a second sub-aperture defined by a second sub-array of the ultrasound transducers; and
generating an average signal, corresponding to the focal point, wherein the generating comprises:
generating a full aperture signal by coherently adding the first sub-aperture signal and the second sub-aperture signal;
generating a first sub-aperture logarithmic signal by logarithmically detecting the first sub-aperture signal;
generating a second sub-aperture logarithmic signal by logarithmically detecting the second sub-aperture signal;
generating a full aperture logarithmic signal by logarithmically detecting the full aperture signal; and
averaging the first sub-aperture logarithmic signal, the second sub-aperture logarithmic signal, and the full aperture logarithmic signal to produce the average signal corresponding to the focal point.

2. The method according to claim 1, further comprising:
converting ultrasound waves, that are incident upon the first sub-aperture, to a first set of ultrasound signals, and
converting ultrasound waves, that are incident upon the second sub-aperture, to a second set of ultrasound signals; and
generating, by beamforming the first set of ultrasound signals, the first sub-aperture signal that corresponds to the focal point; and
generating, by beamforming the second set of ultrasound signals, the second sub-aperture signal that corresponds to the focal point;

3. The method according to claim 1, further comprising:
generating an ultrasound image by at least one selected from a group consisting of:
iteratively repeating the generating of the average signal at multiple different focal points; and
generating, in parallel, the average signal at multiple different focal points.

4. The method according to claim 1, further comprising:
transmitting, from the first sub-aperture, a transmitted first sub-aperture signal;
transmitting, from the second sub-aperture, a transmitted second sub-aperture signal;
generating a transmitted full aperture signal by coherently adding the transmitted first sub-aperture signal and the transmitted second sub-aperture signal, wherein
the obtaining of the beamformed first sub-aperture signal and the beamformed second sub-aperture signal is performed for both the transmitted first sub-aperture signal and transmitted second sub-aperture signal, and
the generating of the average signal is performed for each of the transmitted first sub-aperture signal, the transmitted second sub-aperture signal, and the transmitted full aperture signal

5. The method according to claim 1, wherein the first sub-array and the second sub-array each comprise ultrasound transducers that are consecutive in the lateral direction, such that the first sub-aperture and the second sub-aperture are each spatially continuous in the lateral direction.

6. The method according to claim 5, wherein
the first sub-aperture is disposed entirely on a first side of a center of the array of ultrasound transducers in the lateral direction,
the second sub-aperture is disposed entirely on a second side of the center of the array of ultrasound transducers in the lateral direction.

7. The method according to claim 1, wherein
the first sub-array and the second sub-array each comprise non-consecutive groups of ultrasound transducers, such that the first sub-aperture and the second sub-aperture are spatially intermittent in the lateral direction.

8. The method according to claim 7, wherein
the non-consecutive groups of the first sub-array and the non-consecutive groups of the second sub-array are interleaved such that the spatially intermittent first sub-aperture and the spatially intermittent second sub-aperture overlap in the lateral direction.

9. The method according to claim 1, wherein each of the ultrasound transducers is at least one selected from a group consisting of a capacitive micromachined ultrasound transducer (CMUT) and a piezoelectric micromachined ultrasonic transducer (PMUT).

10. The method according to claim 1, wherein the array of ultrasound transducers is a two-dimensional array comprising rows of ultrasound transducers, the rows being distributed in the lateral direction.

11. A non-transitory computer readable medium (CRM) storing computer readable program code for reducing speckle in ultrasound data corresponding to ultrasound waves received by an array of ultrasound transducers, the computer-readable program code causing a computer to:
obtain a first sub-aperture signal and a second sub-aperture signal, each corresponding to a focal point, wherein
the first sub-aperture signal corresponds to a first sub-aperture defined by a first sub-array of the ultrasound transducers, and
the second sub-aperture signal corresponds to a second sub-aperture defined by a second sub-array of the ultrasound transducers; and
generate an average signal, corresponding to the focal point, wherein the generating comprises:
generating a full aperture signal by coherently adding the first sub-aperture signal and the second sub-aperture signal;
generating a first sub-aperture logarithmic signal by logarithmically detecting the first sub-aperture signal;
generating a second sub-aperture logarithmic signal by logarithmically detecting the second sub-aperture signal;
generating a full aperture logarithmic signal by logarithmically detecting the full aperture signal; and
averaging the first sub-aperture logarithmic signal, the second sub-aperture logarithmic signal, and the full aperture logarithmic signal to produce the average signal corresponding to the focal point.

12. The non-transitory CRM of claim 11, wherein the computer-readable program code further causes the computer to:
generate an ultrasound image by at least one selected from a group consisting of:
iteratively repeating the generating of the average signal at multiple different focal points, and
generating, in parallel, the average signal at multiple different focal points.

13. The non-transitory CRM of claim 11, wherein the computer-readable program code further causes the computer to:
generate a transmitted full aperture signal by coherently adding a transmitted first sub-aperture signal and a transmitted second sub-aperture signal, wherein
the transmitted first sub-aperture signal is transmitted from the first sub-aperture, and
the transmitted second sub-aperture signal is transmitted from the second sub-aperture.

14. An ultrasound system for reducing speckle in ultrasound data by aperture compounding, the ultrasound system comprising:
an array of ultrasound transducers, distributed in a lateral direction, that includes:
a first sub-array of the ultrasound transducers, defining a first sub-aperture, that converts ultrasound waves, incident upon the first sub-aperture, to a first set of ultrasound signals; and
a second sub-array of the ultrasound transducers, defining a second sub-aperture, that converts ultrasound waves, incident upon the second sub-aperture, to a second set of ultrasound signals;
electronic circuitry, comprising:
a first beamformer, coupled to the first sub-array, that beamforms the first set of ultrasound signals to generate a first sub-aperture signal corresponding to a focal point;
a second beamformer, coupled to the second sub-array, that beamforms the second set of ultrasound signals to generate a second sub-aperture signal corresponding to the focal point; and
a processor that:
generates an average signal, corresponding to the focal point, wherein the generating comprises:
generating a full aperture signal by coherently adding the first sub-aperture signal and the second sub-aperture signal,
generating a first sub-aperture logarithmic signal by logarithmically detecting the first sub-aperture signal,
generating a second sub-aperture logarithmic signal by logarithmically detecting the second sub-aperture signal,
generating a full aperture logarithmic signal by logarithmically detecting the full aperture signal, and
averaging the first sub-aperture logarithmic signal, the second sub-aperture logarithmic signal, and the full aperture logarithmic signal to produce the average signal corresponding to the focal point.

15. The ultrasound system according to claim 14, wherein the processor:
generates an ultrasound image by at least one selected from a group consisting of:
iteratively repeating the generating of the average signal at multiple different focal points, and
generating, in parallel, the average signal at multiple different focal points.

16. The ultrasound system according to claim 14, wherein
the first sub-array transmits, from the first sub-aperture, a transmitted first sub-aperture signal,
the second sub-array transmits, from the second sub-aperture, a transmitted second sub-aperture signal,
the first beamformer and the second beamformer each perform the beamforming for each of the transmitted first sub-aperture signal and the transmitted second sub-aperture signal,
the processor generates a transmitted full aperture signal by coherently adding the transmitted first sub-aperture signal and the transmitted second sub-aperture signal, and
the processor performs the generating of the average signal for each of the transmitted first sub-aperture signal, the transmitted second sub-aperture signal, and the transmitted full aperture signal.

17. The ultrasound system according to claim 14, wherein the first sub-array and the second sub-array each comprise ultrasound transducers that are consecutive in the lateral direction, such that the first sub-aperture and the second sub-aperture are each spatially continuous in the lateral direction.

18. The ultrasound system according to claim 17, wherein
the first sub-aperture is disposed entirely on a first side of a center of the array of ultrasound transducers in the lateral direction,
the second sub-aperture is disposed entirely on a second side of the center of the array of ultrasound transducers in the lateral direction.

19. The ultrasound system according to claim 14, wherein
the first sub-array and the second sub-array each comprise non-consecutive groups of ultrasound transducers, such that the first sub-aperture and the second sub-aperture are spatially intermittent in the lateral direction.

20. The ultrasound system according to claim 19, wherein
the non-consecutive groups of the first sub-array and the non-consecutive groups of the second sub-array are interleaved such that the spatially intermittent first sub-aperture and the spatially intermittent second sub-aperture overlap in the lateral direction.

21. The ultrasound system according to claim 14, wherein each of the ultrasound transducers is at least one selected from a group consisting of a capacitive micromachined ultrasound transducer (CMUT) and a piezoelectric micromachined ultrasonic transducer (PMUT).

22. The ultrasound system according to claim 14, wherein the array of ultrasound transducers is a two-dimensional array comprising rows of ultrasound transducers, the rows being distributed in the lateral direction.

23. The ultrasound system according to claim 14, further comprising:
a handheld ultrasound probe, comprising:
the array of ultrasound transducers, and
the electronic circuitry; and
a processing device, being one selected from a group consisting of a computer, a tablet, and a smartphone, the processing device comprising:
the processor.
